(19) United States
(12) Patent Application Publication
(10) Pub. No.: US 2013/0196349 A1
(43) Pub. Date: Aug. 1, 2013

(54) In Vitro Tumor Metastasis Model

(71) Applicants: George A. Martin, Berkeley, CA (US); Lee A. Noll, Whitmore Lake, MI (US); William P. Pfund, Portage, MI (US)

(72) Inventors: George A. Martin, Berkeley, CA (US); Lee A. Noll, Whitmore Lake, MI (US); William P. Pfund, Portage, MI (US)

(73) Assignees: REALBIO TECHNOLOGY, INC., Kalamazoo, MI (US); ROCHE MOLECULAR SYSTEMS, INC., Pleasanton, CA (US)

(21) Appl. No.: 13/564,997

(22) Filed: Aug. 2, 2012

Related U.S. Application Data
(60) Provisional application No. 61/514,291, filed on Aug. 2, 2011.

(51) Int. Cl. C12Q 1/04; C12Q 1/02; G01N 33/68; C12M 1/12; C12N 5/09

(52) U.S. Cl. CPC C12Q 1/04; C12M 25/14; C12N 5/0693; G01N 33/6893; C12Q 1/025
USPC 435/7.23; 435/289.1; 435/297.1; 435/325; 435/395; 435/34

(57) ABSTRACT

This invention provides a system and methods for modeling tumor metastasis in vitro where primary tumor tissue is cultivated in an orientation and an environment such that the natural composition, three-dimensional organization, and environmental conditions of the tumor can be adjusted. The invention further provides mechanism for inducing tumors to undergo metastatic processes resulting in production of tumor progenitor or stem cells that can be collected, characterized, or used to induce tumors in normal tissue constructs in vitro.

### FIELD OF THE INVENTION

This invention relates to the field of cancer biology. More specifically, the invention relates to in vitro systems for culturing cancer cells and tissues.

### BACKGROUND OF THE INVENTION

The majority of cancer deaths are due to complications from metastasis to distal organs. Developing an in vitro model that is representative of the metastasis process will significantly expedite research of the molecular events associated with metastasis and will contribute to the development of novel therapies that target metastatic processes.

The current standard for metastatic tumor models is represented by in vivo models (xenografts) involving mice, rats or other animal species commonly employed for medical research. These models suffer from a number of significant drawbacks including low and unpredictable rates of tumor engraftment and the confounding influences of non-human molecular factors from the host species, see e.g. Quintana et al., (2008), Efficient tumor formation by single human melanoma cells, Nature 456(7222):593. Both in vivo and traditional in vitro models also have the drawback of losing human stromal cells normally associated with the tumor, see e.g., Montel et al., (2006) Tumor-stromal interactions reciprocally modulate gene expression patterns during carcinogenesis and metastasis. Int. J. Cancer, 119:25. There is also difficulty in identifying and isolating any metastatic cancer stem cells that may be released by tumors into the complex milieu of the host animal's circulatory system, see e.g. Talmadge and Fidler, (2010) AACR Centennial Series: The Biology of Cancer Metastasis: Historical Perspective, Cancer Res; 70(14):5649.

Another reason aspect for switching to in vitro models for studying metastasis is ethical considerations. If good alternatives to animal models are available they are preferred. The European Union Directive 2010/63/EU on the protection of animals used for scientific purposes states that it is: “essential, both on moral and scientific grounds, to ensure that each use of an animal is carefully evaluated as to the scientific or educational validity, usefulness and relevance of the expected result of that use. The likely harm to the animal should be balanced against the expected benefits of the project.” Furthermore, there are many documented cases were animal models fail to accurately predict medical outcomes in humans. See e.g., Knight, A. (2007) Systematic reviews of animal experiments demonstrate poor human clinical and toxicological utility. Altern. Lab. Anim. 35:641; Gura T. (1997) Systems for identifying new drugs are often faulty. Science 278:1041.

Traditional in vitro methods for studying metastasis typically involve the use of tumor cell lines cultivated in two-dimensional systems with little ability to simulate gradients of nutrients, gases, and other factors present in the natural in vivo environment. These cell line-based systems lack the mixed cell populations, the natural three-dimensional arrangements of cells, or the varied micro-environments present in tumors in vivo. As a result of the limitations described above, both in vivo and in vitro methods currently in use lack “biorelevance” and are poorly predictive of natural tumor behavior.

### SUMMARY OF THE INVENTION

This invention provides a culture system and methods for modeling tumor metastasis in vitro where the tumor tissue is cultivated in an orientation and in an environment such that the natural composition, three-dimensional organization, and environmental conditions of the tumor can be simulated and controlled. The invention further provides mechanisms for inducing tumors to undergo metastatic processes resulting in production of tumor progenitor or stem cells that can be collected, characterized, or used to induce tumors in normal tissue constructs in vitro.

In some embodiments, the invention is a combination of a three-dimensional bioreactor and one or more tumor cells in which the system parameters are such that the tumor cells maintain their normal metastatic potential. In variations of this embodiment, the bioreactor comprises a cell-supporting but cell-permeable matrix separating at least two fluid chambers with fluid flowing therethrough and at least one gas chamber connected to each of the fluid chambers and the tumor cells have been introduced into one of the at least two fluid chambers containing suitable nutrient medium and gas sufficient to sustain tumor growth. In further variations of this embodiment, the metastatic cells include cells having characteristics of circulating tumor cells and circulating tumor progenitor cells including the presence of one or more of the following: EpCAM, cytokeratins (CK) 5, 7, 18 and 19, IGF-1R, BCL2, HER2, EphB4, CA19-9, CEA, CD133, MUC1, Survivin, PTEN, CD44v6, N-cadherin, and FAP (Seprase).

In another embodiment, the invention is a method of generating metastatic tumor cells in vitro comprising: introducing one or more tumor cells into a bioreactor; providing to the bioreactor fluid culture media and gas composition supportive of growth of the tumor cells; incubating the bioreactor under conditions and for a time sufficient for the tumor cells to proliferate and produce metastatic cells; and collecting metastatic tumor cells. In variations of this embodiment, the bioreactor comprises a cell-supporting but cell-permeable matrix separating at least two fluid chambers with fluid flowing therethrough and at least one gas chamber connected to each of the fluid chambers. In further variations of this embodiment, the tumor cells are introduced into the first fluid chamber and metastatic tumor cells are collected from among cells that have migrated into the second chamber.

In yet another embodiment, the invention is a method of manipulating a culture of tumor cells in a bioreactor to reveal or alter metastatic potential of the tumor cells. In variations of this embodiment, the bioreactor comprises a cell-supporting but cell-permeable matrix separating at least two fluid chambers with fluid flowing therethrough and at least one gas chamber connected to each of the fluid chambers and said tumor cells have been introduced into one of the at least two fluid chambers containing suitable nutrient medium and gas sufficient to sustain tumor growth. In further variations of this embodiment, manipulating the culture comprises altering of one or more of suitable nutrient concentration, oxygen concentration and acidity or comprises administering one or more of test compounds, antibodies or biologics.

In yet another embodiment, the invention is a method of identifying an agent capable of inhibiting or stimulating tumor metastasis comprising: preparing a suspension of cells derived from a tumor; introducing a sample of the suspension into a first fluid chamber of a bioreactor, the bioreactor, comprising a cell-supporting but cell-permeable matrix separating at least two fluid chambers with fluid flowing therethrough and at least one gas chamber connected to each of the fluid chambers; supplying to said at least two fluid chambers fluid culture media and gas composition suitable to support tumor growth; incubating the bioreactor under conditions and for a time sufficient for cell proliferation and formation of metastatic cells; introducing a candidate agent into the first or the second chamber; collecting cells that have migrated into the second chamber; identifying and monitoring the fraction of metastatic tumor cells among the collected cells. In variations of this embodiment, the agent is selected from among a small-molecule compound, an antibody or a biologic.

In yet another embodiment, the invention is a method of assessing metastatic potential of a tumor comprising: preparing a suspension of cells derived from the tumor; introducing a sample of the suspension into a first fluid chamber of a bioreactor, the bioreactor, comprising a cell-supporting but cell-permeable matrix separating at least two fluid chambers with fluid flowing therethrough and at least one gas chamber connected to each of the fluid chambers; supplying fluid culture media and gas composition supportive of tumor growth to said at least two fluid chambers; incubating the bioreactor under conditions and for a time sufficient for cell proliferation and production of metastatic cells; collecting cells that have migrated into the second chamber and identifying metastatic tumor cells among the collected cells.

### BRIEF DESCRIPTION OF THE FIGURES

FIG. 1 is a microscopic image of cell culture produced in Example 1.

FIG. 2 shows glucose consumption by the cell cultures produced in Example 2.

FIG. 3 shows lactic acid production by the cell cultures produced in Example 2.

FIG. 4 shows the rate of cell migration out of the cultures produced in Example 2.

FIG. 5 shows CTC and CTPC production (including as proportion of viable cells) in the cultures of Example 2.

FIG. 6 shows glucose consumption by the cell cultures produced in Example 3.

FIG. 7 shows lactic acid production by the cell cultures produced in Example 3.

FIG. 8 shows CTC production by the cultures in Example 3.

FIG. 9 shows CTPC production by the cultures in Example 3.

FIG. 10 shows relative CTC production by the cultures in Example 3.

FIG. 11 shows relative CTPC production by the cultures in Example 3.

FIG. 12 shows glucose consumption by the cell cultures produced in Example 4.

FIG. 13 shows lactic acid production by the cell cultures produced in Example 4.

FIG. 14 shows the rate of cell migration out of the cultures produced in Example 4.

FIG. 15 shows the relative rate of CTC and CTPC production by the cultures in Example 4.

FIG. 16 shows metabolic profile of Capan-2 cell line grown in Example 5.

FIG. 17 shows metabolic profile of MIA PaCa-2 cell line grown in Example 5.

FIG. 18 shows the rate of cell migration out of the cultures produced in Example 5.

### DETAILED DESCRIPTION OF THE INVENTION

### Definitions

To facilitate the understanding of this disclosure, the following definitions of the terms used herein are provided.

The term “RealBio D4™ Culture System” is a trade name of a bioreactor marketed by RealBio® Technology, Inc. (Kalamazoo, Mich.).

The term “bioreactor” refers to a device that supports a biologically active environment wherein cells or tissues can be grown ex vivo.

The term “cancer cells” and “tumor cells” are used interchangeably to refer to cells derived from a cancer or a tumor, or from a tumor cell line or a tumor cell culture.

The term “metastatic cells” or “metastatic tumor cells” refers to the cells that have the ability to produce a metastasis.

The term “stem cells” refers to multi-potent or pluripotent cells capable of getting rise to many other cell types.

The term “progenitor cells” refers to undifferentiated cells destined to produce a specific cell type.

The term “circulating tumor cells” or “CTCs” refers to tumor cells found in circulation of a patient having a tumor. This term typically does not include hematological tumors where the majority of the tumor is found in circulation.

The term “circulating tumor progenitor cells” or “CTPCs” refers to tumor cells found in circulation of a patient having a tumor that are not yet fully differentiated to the point of expressing all characteristics of mature tumor cells.

The term “cancer stem cells” refers to cells found within tumors that possess characteristics associated with normal stem cells including their ability to give rise to all cell types found in a particular tumor sample.

The term “matrix” or “scaffold” are used interchangeably to refer to solid material that provides support for cells and tissues growing in a bioreactor.

The term “primary tumor” refers to a tumor growing at the site of the cancer origin.

The term “metastatic tumor” refers to a secondary tumor growing at the site different from the site of the cancer origin.

The term “migration” means observable displacement of cells in a three-dimensional space. As used herein, “migration” means both active migration as well as passive migration of cells.

The term “cell line” refers to a population of cells that through cell culture, has acquired the ability to proliferate indefinitely in vitro.

The term “primary cell culture” refers to a cell culture established from an organism in the course of a study. A primary cell culture may or may not give rise to a cell line.

The term “established cell line” refers to a cell line propagated in vitro multiple times prior to a study.

The term “metabolic parameter” refers to a parameter reflective of the metabolism of the cells in a culture.

The term “biomarker” refers to a biological marker characterizing a phenotype. A biomarker typically includes a gene or a gene product. Depending on the gene, “detecting a biomarker” may include detecting altered gene expression, epigenetic modifications, germ-line or somatic mutations, etc. In case of a gene product, “detecting a biomarker” may mean detecting the presence, quantity or change in quantity of a cell surface marker, a soluble compound such as cytokine, etc. “Detecting a biomarker” may also include detecting a metabolite reflective of a gene's expression or activity.

The term “tumor biomarker” or “cancer biomarker” refers to a biomarker characteristic of a tumor or cancer but not normal tissue.

The term “small molecule” or “small-molecule compound” refers to a low molecular weight non-polymeric organic compound that has (or is being tested for having) beneficial pharmacological and therapeutic properties typically including binding with high affinity to a biopolymer such as protein, nucleic acid or a polysaccharide and altering the activity or function of the biopolymer. The upper molecular weight limit for a small molecule is approximately 800 Daltons.

The term “biologic” refers to a biologic medical product that has (or is being tested for having) beneficial pharmacological and therapeutic properties that has been created by a biological process rather than chemically synthesized. Biologics include for example, blood components, living cells and recombinant proteins.

The invention provides a bioreactor with mixed populations of cancer cells (i.e., tumor culture) under appropriate system parameters for growing tumor tissues in a three-dimensional arrangement replicating the tumor state in vivo. More particularly, the mixed cancer cell populations are grown in such a manner that they maintain the metastatic potential existing in vivo so that small changes in the system parameters can stimulate or suppress release of metastatic cells. Release of these cells may be stimulated or suppressed by exposing the mixed cancer cell cultures to metastasis triggers such as hypoxia, nutrient deprivation, changes in acidity and other biological or chemical stimulants, or by exposing the mixed cancer cell cultures to metastasis inhibitors. In the course of the culture growth, cells migrating out of the culture into the circulating medium may be collected, counted and analyzed for metastatic potential. Similarly, after a change in system parameters, the migrating cells may be collected and analyzed to measure the effect of the change on metastasis.

The bioreactor used in the present invention supports continuous production and output of tumor cells and possibly, metastatic tumor cells over extended periods of time, up to several months. A suitable bioreactor is typically composed of a matrix or scaffold for cell attachment or immobilization; one or more fluid chambers bathing the cell scaffold from above and below while allowing metabolic gases to diffuse; and one or more gas chambers for supplying gases to the fluid chambers.

In some embodiments, the bioreactor comprises two fluid chambers separated by a matrix for receiving cells, wherein the cells are seeded in one chamber, and each fluid chamber is connected to a gas chamber. In some embodiments, the first and second fluid chambers of the bioreactor are configured to flow the first and second fluids respectively tangentially to the surface of the matrix material. In some embodiments, the first and second gassing chambers of the bioreactor are operably linked to the first and second fluid chambers providing gas to the fluid chambers. In some embodiments, each gassing chamber is separated from the fluid chamber by a gas permeable membrane positioned between the fluid chamber and the gas chamber.

An example of a suitable device is provided in U.S. Pat. No. 7,682,822, which is hereby incorporated in its entirety, by reference. In some embodiments, the bioreactor is the RealBio D4™ Culture System (RealBio Technology, Inc., Kalamazoo, Mich.) The RealBio D4™ Culture System is a bioreactor designed to recreate a natural, in vivo-like environment for culturing cells. The bioreactor is used to create “ex vivo generated tissue” or a three-dimensional culture of cells that mimics biological properties of naturally occurring tissue such as for example, normal liver, kidney, gastrointestinal, respiratory, cardiac, adipose, and skin tissues as well as tumors derived from these tissues. The bioreactor combines an open three-dimensional cell scaffold or matrix, perfused nutrient medium, and a mechanism for controlling metabolic gas exchange decoupled from nutrient delivery. Combined, these features allow researchers to establish in vivo-like nutrient and gas gradients across the cultured tissues.

In one embodiment, the bioreactor used in the present invention utilizes a three-dimensional matrix to create and maintain a mixed population of cells simulating a tumor found in a human or other mammalian body. Tumors include without limitation, melanoma, hereditary non-polyposis colorectal cancer (HNPCC) tumors, nervous system tumors such as neuroblastoma, glioblastoma and retinoblastoma, various carcinomas including colon, gastric, pancreatic, renal, ovarian, prostate, breast, cervical, medullary and papillary thyroid carcinoma, non-small cell lung carcinoma (NSCLC) and adenocarcinoma and various sarcomas including rhabdomyosarcoma and osteosarcoma. In addition, metastatic tumors that have developed from various primary tumors are also included in the scope of the present invention.

The matrix can be manufactured from an inert material such as polystyrenes, polycarbonates or polyesters, including biodegradable polyesters such as, e.g., polycaprolactone. Other examples of suitable matrix materials include plastic, glass, ceramic or natural biomatrix materials such as collagen, alginates, proteoglycans and laminin. The three-dimensional matrix may be manufactured from one or both of non-woven and woven fibers, having an ordered or random fiber arrangement. An example of a suitable non-woven fabric having a random fiber arrangement is polyester material such as a felt fabric formed from polyethylene terephthalate (PET). In some embodiments, the matrix member is a three dimensional matrix manufactured from a polyester fiber, which has a random fiber arrangement. In some embodiments, the matrix may have pores of any size suitable to permit the three-dimensional growth while also permitting cells to migrate through the matrix.

Thickness and density of matrix fibers and the size of pores optimal for each tumor and cell type may be selected empirically. In certain embodiments, the thickness of a matrix member ranges from about 0.1 to about 3 mm. The matrix may have pores ranging in size from about 10 to about 300 microns.

In some embodiments, the invention comprises the use of a bioreactor to establish a three-dimensional tumor culture that has retained its natural ability to metastasize, i.e. shed metastatic cells (including CTC and CTPC). The tumor culture is established from tumor cells. In this embodiment, the tumor cells may be obtained from primary or metastatic tumors obtained directly from patients or as commercially available xenografts. In addition, tumor cells may be obtained from primary tumor cultures or established tumor cell lines. Solid tumors may be processed by either mechanical or complete or partial enzymatic or chemical dissociation or a combination of these techniques until a suspension of single cells or multi-cell tissue fragments of desired size is obtained. Enzymatic digestion may be carried out by a combination of one or more proteases and nucleases known in the art. After processing, the seeding suspension of cells or multi-cell tissue fragments is introduced into the bioreactor. One or more cells seeded into the bioreactor may represent one or more cell types present in a tumor.

In variations of this embodiment, the bioreactor may be prepared to receive the seeding suspension. For example, the bioreactor may be equilibrated by perfusion with nutrient medium and gases. In some embodiments, the bioreactor is equilibrated to typical conditions for culturing human cells: 37° C. and 5% CO₂. In some embodiments, the matrix may be pre-treated with cell attachment factors such as collagen or laminin. After equilibration, the bioreactor may be seeded with the seeding suspension. In some embodiments, the suspension is introduced into RealBio D4™ Culture System.

In some embodiments, the invention comprises the use of a bioreactor to maintain and propagate a three-dimensional tumor culture while the tumor continues to metastasize, i.e. shed metastatic progenitor cells (including CTCs and CTPCs). In variations of this embodiment, after the bioreactor is seeded with a seeding suspension, the bioreactor may be retained in desired orientation optionally without perfusion to allow cells to settle into the culture scaffold. After the settling period, the bioreactor may be repositioned in a different orientation. In some embodiments, the bioreactor may be placed on an incline to facilitate separation of non-adherent cells by gravity. After the settling period, medium flow may also be initiated. In some embodiments, the bioreactor may be placed on an incline and pulsed medium flow may be initiated. In some embodiments, the cultures are maintained in the RealBio D4™ Culture System placed on a 45° incline with a pulsed medium flow cycle.

Cultures may be monitored to confirm growth and tumor expansion. As described in greater detail below, the growth may be monitored e.g. by measuring the increase in the rate of nutrient utilization or waste production. In some embodiments, the growth is monitored by measuring the rate of glucose consumption or lactate production. In further embodiments, the growth may be monitored by measuring concentration of additional metabolites including e.g., glutamine, urea, bicarbonate, ammonia, amino acids, lipids, proteins and sugars. The growth may also be monitored by withdrawing samples of tumors to determine viable cell count by any of the techniques known in the art. The cultures may be continued for several days, weeks or months.

The invention allows for generation of enriched populations of metastatic cells for subsequent study. Tumors in vivo generate and release metastatic cells (including CTCs and CTPCs). In circulation, metastatic cells become diluted by the large volume of blood and body fluids such as lymph. These cells are very rare compared to normal cells in circulation. In contrast, the bioreactor has a much smaller volume from which the sloughed cells are collected. Furthermore, the bioreactor does not contain additional cell types, e.g. white and red blood cells normally present in circulation alongside with metastatic cells. Thus in the bioreactor, the concentration of released metastatic cells is much higher and they can be collected much easier from relatively small tumor specimens without the need for high efficiency cell separation technologies.

In some embodiments, the sloughed cell population comprising metastatic cells may be continually or periodically removed from the bioreactor. The cells may be removed via a harvest port engineered into a fluid chamber of the bioreactor.

In some embodiments, the invention comprises the use of a bioreactor to harvest and analyze metastatic cells including circulating tumor cells (CTCs) and circulating tumor progenitor cells (CTPCs). In this embodiment, samples of sloughed cells are taken at different stages may be analyzed and compared. In some embodiments, a sample of whole blood from the animal or patient bearing the tumor used to initiate the cultures, a sample of the dissociated tumor suspension used to seed the culture systems, and the samples collected from the bioreactor may be analyzed and compared. Furthermore, upon termination of the culture, the matrix or scaffold may be excised from the bioreactor for examination of tissue development by direct staining, traditional histological processing and scanning electron microscopy (SEM). For microscopic analysis the sample may be stained for example, with hematoxylin and eosin (H&E) or other differential stains, e.g., PROTOCOL® HEMA 3 staining. All cells may be stained with the fluorescent nucleic acid-binding dye, such as Hoechst 33342 or DAPI to aid in differentiating cells from cellular debris. Cells exhibiting positive staining with the various markers described below may be identified as CTCs or CTPCs, counted and further characterized.

Several biomarkers as well as morphological, immunological and physiological tests or combinations thereof may be used to identify CTCs and CTPCs. See e.g. Sun et al. (2011), Circulating tumor cells: advances in detection methods, biological issues, and clinical relevance, J. Cancer Res. Clin. Oncol. 137:1151-1173; Man, et al. (2011), Currently used markers for CTC isolation—advantages, limitations and impact on cancer prognosis, J. Clin. Exper. Pathol. 1:1. For example, CTCs and CTPCs may be identified by their ability to adhere to cell adhesion molecules (CAM), as well as by the presence of certain specific biomarkers including EpCAM, cytokeratins (CK) 5, 7, 18 and 19. Depending on the tumor of origin, CTCs may also be identified based on the presence of tumor-specific biomarkers including IGF-1R, BCL2, HER2, EphB4, CA19-9, CEA, CD133, MUC1, Survivin and PTEN. For example, CTCs originating from the pancreas would exhibit positive staining with standard epithelial markers and human pancreatic tumor markers (EpCAM and CA19-9). CTPCs may be identified in a similar fashion except that tumor progenitor markers (CD44v6, N-cadherin, and FAP (Seprase)) may be used in the place of epithelial markers. In some embodiments of the invention, CTCs and CTPCs are identified using VITA-ASSAY™ AR16 platform (Vitatex, Inc., Stony Brook, N.Y.).

In some embodiments, the invention comprises the use of a bioreactor for testing the collected cells for their capacity to form metastatic lesions in healthy tissues. This embodiment may further comprise studying the processes related to the development of metastases. In this embodiment, collected cells may be infused into additional bioreactors in which cultures of mixed cell populations representing healthy “target” tissues have been established. In some embodiments, the study of the metastatic process comprises characterizing cells collected following changes in system parameters (e.g. changes in oxygen concentration, pH, nutrients etc.) by genetic analysis (e.g. for the presence of biomarkers described above), in vitro invasion assays, cell marker-based tumor progenitor identification assays, anti-cancer drug response assays, as well as other established methods for identifying and characterizing metastatic cells. Samples of the circulating medium may also be analyzed directly for changes in soluble metastasis-related biomarkers in response to changes in system parameters.

The invention allows the effects of single or combinations of culture parameters to be studied. For example, the nutrient and oxygen levels in the system may be dropped in concert to simulate conditions that are thought to stimulate metastatic behavior in large tumors. In some embodiments, ports are integrated into one or more of the fluid chamber inputs to deliver liquid components to the bioreactor. In addition to nutrients, anti-metastasis agents, immunological factors and anti-cancer compounds and biologics, including compounds that modulate gene expression and cell function such as cytokines, toxins, nucleic acids (e.g., microRNA), or other cell types may also be added to the perfusion fluids of the bioreactor to study the effect of treatment with these compounds as single agents or combinations.

In some embodiments, the invention comprises use of a bioreactor to create an in vitro model of a patient's cancer wherein the cancer has retained its metastatic potential which can aid in the selection of personalized anti-cancer treatments that would prevent or eliminate metastases in the patient.

In some embodiments, the invention comprises the use of a bioreactor to determine physiological characteristics of a tumor culture in relation to the tumor's ability to metastasize. In this embodiment, the recoverable suspension of the tumor culture maintained in the bioreactor may be periodically sampled and analyzed. In other variations of this embodiment, a sample may be retrieved from a compartment separated from the tumor culture for example, by the matrix separating the chambers of the bioreactor, such as the bottom chamber. In some embodiments, the sample is withdrawn from the bottom compartment of the RealBio D4™ Culture System. The analysis of culture parameters (including glucose and lactate concentration, migrating cell numbers) may be performed at this time. Glucose concentration may be measured using any technique and device known the art, for example using ACCU-CHEK® Aviva blood glucose monitor (Roche Diagnostics Corp., Indianapolis, Ind.). Lactate similarly may be measured using any technique and device known the art, for example using the Lactate Plus test meter (Nova Biomedical Corp., Waltham, Mass.). The total cell density of each sample may also be estimated using any technique and device known the art, for example using the TC10™ Automated Cell Counter (Bio-Rad Labs., Hercules, Cal.).

According to the present invention, tumors can be cultured in the bioreactor in any physiologically acceptable liquid culture medium. Guidance for selecting culture medium and conditions may be found in Sandell, L. and Sakai, D. Mammalian Cell Culture. Current Protocols Essential Laboratory Techniques 5:4.3.1-4.3.32, John Wiley & Sons, 2011. A medium optimal for a particular tumor type may be empirically found among the many commercially available products including AIM V, IMDM, MEM, DMEM, RPMI 1640, Alpha Medium or McCoy's Medium. The medium can be supplemented with serum as known in the art, typically at 1% to 50%. Alternatively serum substitutes comprising serum albumin, cholesterol, lecithin and inorganic salts may be used. The tumor cultures are typically carried out at a pH which approximates physiological conditions, between 6.9 and 7.4. The medium is typically exposed to an oxygen-containing atmosphere which contains from 2 to 20% oxygen. In some embodiments, the parameters are altered to simulate hypoxia, acidosis, nutrient starvation, accumulation of waste products and other pathological conditions known to occur in tumors.

The invention further includes a method and system for selecting and testing anti-tumor and anti-metastasis agents including compounds, antibodies and biologics. Such candidate agents may be introduced into the bioreactor cultures of the present invention and tested for their ability to alter the production of metastatic cells (including CTCs and CTPCs) by the cultures.

The invention provides the flexibility to present different nutrient and gas conditions on each side of the cultured tumor tissue in the bioreactor. In some embodiments, independently, oxygen-rich and oxygen-poor gases may be supplied to the two sides of the culture system. Since manipulation of such nutrient and gas gradients is necessary for gaining better understanding of the role that the tumor microenvironment plays in tumor development, growth and metastasis; and since these gradients cannot be readily established and manipulated using other in vitro technologies or within in vivo studies; this feature of the invention provides a significant advantage over existing technologies.

### EXAMPLES

### Example 1

Establishment of a Metastatic Pancreatic Tumor In Vitro

A metastatic pancreatic tumor (pancreas to liver) weighing approximately 1 gram was minced and partially dissociated enzymatically before approximately ⅕ of the dissociated mass was infused into a RealBio D4™ Culture System bioreactor. The mixed population of tumor and associated stromal cells was maintained in the bioreactor by circulating Iscove's Modified Dulbecco's Medium (IMDM) supplemented with fetal bovine serum (FBS) (10%) and antibiotics through both the upper and lower fluid chamber. The bioreactor was maintained in an incubator at 37° C. with a 5% CO₂ environment. Samples of the culture medium were collected from the lower compartment of the bioreactor three times per week to count the number of cells shed by the cultured tumor and to monitor metabolic activity of the culture (glucose consumption and lactate production). After 29 days in culture, a sample of the cells migrating out of the cultured tumor was analyzed to identify and enumerate circulating tumor cells (CTCs) and circulating tumor progenitor cells (CTPCs) using VITA-ASSAY™ AR16 platform (Vitatex, Inc., Stony Brook, N.Y.). Scanning electron microscopy (SEM) was used to examine the cultured tumors from which the CTCs and CTPCs have migrated.

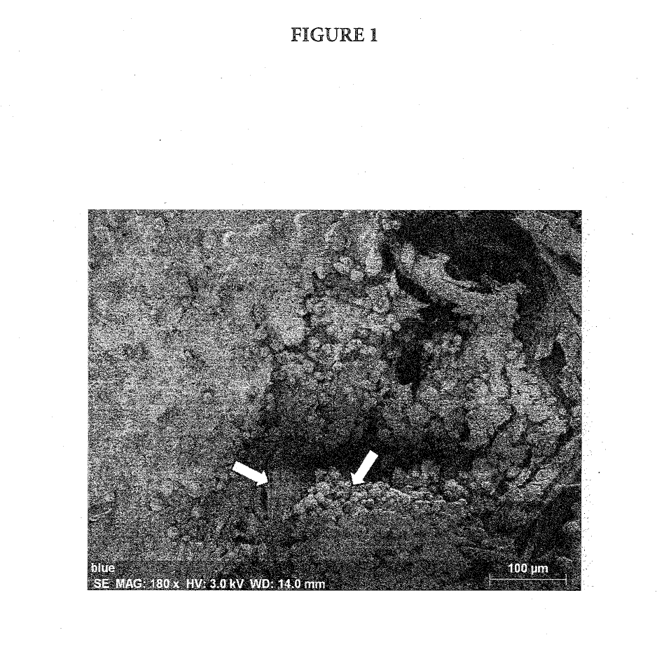

The results are shown on Table 1 and FIG. 1. The sample which included a mixture of live and dead cells of tumor and stromal derivation was significantly enriched for CTCs and CTPCs (Table 1) relative to the extremely low frequency of CTCs and CTPCs typically observed in blood in vivo, despite the absence of any treatments aimed at inducing or augmenting release of metastatic cells.

TABLE 1
Relative Abundance of Circulating Pancreatic Cancer Tumor Cells

| Sample | CTCs | CTPCs |
|---|---|---|
| Typical blood from patient w/metastatic cancer | <0.001% | <0.001% |
| (Data from National Pancreas Foundation) | | |
| Metastatic tumor in the RealBio D4 ™ Culture | 2% | 3% |
| System Relative abundance expressed as | | |
| percentage of total cells or cell-like particles | | |

Electron microscopy of the tumor culture 28 days after cells were seeded into the culture chamber (FIG. 1) confirms the persistence of multiple cell types (shown by arrows).

### Example 2

### In Vitro Production of Circulating Tumor Cells (CTCs) by Metastatic Pancreatic Tumor Tissue

Metastatic pancreatic tumor tissue was obtained as a fresh mouse xenograft tumor (P1) from a commercial source. The xenograft tumor was originally derived from a human adenosquamous carcinoma of the pancreas that had metastasized to the liver of a 46 year old female. The tumor was excised from the host animal and shipped overnight on “blue ice” cold packs in serum-free RPMI culture medium containing penicillin and streptomycin. Upon receipt, the entire tumor mass (wet weight=1.21 g) was immediately processed by mechanical and partial enzymatic dissociation using LIBERASE™ and DNase I (Roche Applied Science, Indianapolis, Ind.)

RealBio D4™ Culture System bioreactors configured with a single, recirculating flow loop were primed with 35 mL of complete culture medium and equilibrated overnight in a standard CO₂ incubator (passive gassing) at 37° C., 5% CO₂. A total of six bioreactors representing duplicates of three minor culture chamber variations were used (Table 2). The minor variations between the test groups involved different orientations of a single, woven synthetic scaffold material with or without surface plasma treatment of the scaffold fibers to evaluate the effect of different scaffold conditions on culture establishment.

TABLE 2
RealBio D4 ™ Culture System Bioreactor Configurations

| Test Group | Scaffold Type | Scaffold Comments |
|---|---|---|
| 1 | A | Standard orientation, untreated |
| 2 | A | Inverted, untreated |
| 3 | B | Standard orientation, plasma treated |

For seeding, medium flow was suspended and bioreactors were placed in a horizontal orientation. 3 mL of the dissociated tumor suspension was infused into the top compartment of each bioreactor. After a 24 hr settling time with no medium flow, the bioreactors were placed on a 45° incline and pulsed medium flow was reinitiated. Samples were collected from the bottom compartment of each bioreactor 3 times per week. Approximately 100 μL of each sample was separated for analyzing routine culture parameters (glucose and lactate concentration, migrating cell numbers) while the remainder of each sample was reserved for CTC and CTPC analysis as described below.

One culture from each test group was terminated after 19 days and the remaining culture after 42 days. Upon termination, the fabric scaffold was excised from each bioreactor and divided into sections for examination of tissue development by direct staining, traditional histological processing and scanning electron microscopy (SEM) as described below.

Glucose concentration was measured using the ACCU-CHEK® Aviva blood glucose monitor (Roche Diagnostics Corp., Indianapolis, Ind.) and lactate concentration was measured using the Lactate Plus test meter (Nova Biomedical Corp., Waltham, Mass.). The total cell density of each sample was estimated using the TC10™ Automated Cell Counter (Bio-Rad Labs., Hercules, Cal.) without trypan blue staining.

Identification and enumeration of Circulating Tumor Cells (CTCs) and Circulating Tumor Progenitor Cells (CTPCs) was performed using the VITA-ASSAY™ AR6W platform. The samples analyzed included a sample of whole blood from the mouse bearing the tumor used to initiate the cultures, a sample of the dissociated tumor suspension used to seed the culture systems, and the samples collected periodically from the lower compartment of the culture chambers. For all samples, VITA-ASSAY™ identifies viable CTCs using EpCAM and CA19-9. Viable CTPCs were identified in a similar fashion except that tumor progenitor markers (CD44v6 and FAP (Seprase)) were used in the place of epithelial markers. All cells were also stained with the fluorescent nucleic acid dye Hoechst 33342 to aid in differentiating cells from cellular debris. Cells exhibiting positive staining with the various markers were counted manually under multi-parametric fluorescence microscopy. Sections of the fabric scaffold excised from cultures after termination on days 19 and 42 were processed by staining directly (HEMA 3 stain), or staining with H&E (after paraffin embedding and sectioning), or by SEM.

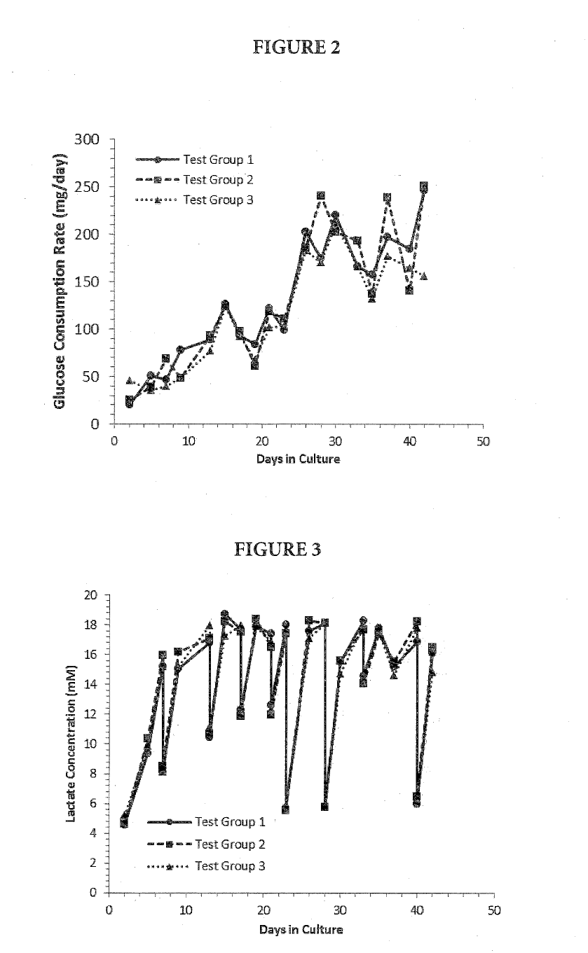

The progress of the cultures was assessed by glucose and lactate analyses shown in FIGS. 2-3. FIG. 2 illustrates the rate of glucose consumption between sampling intervals for each test group defined in Table 2. Values for intervals through Day 19 represent the average from duplicate cultures while values from intervals beyond Day 19 are from single cultures only. Glucose consumption rates increased dramatically and to a similar extent across the entire duration of the study for cultures from all three test groups suggesting that scaffold type and orientation had minimal impact on the rate of culture establishment and expansion (FIG. 2). FIG. 3 illustrates the concentration of lactate in the circulating culture medium for each test group defined in Table 2. Values for time points through Day 19 represent the average from duplicate cultures while values from time points beyond Day 19 are from a single culture only. The vertical drops on days 7, 13, 17, 21, 23, 28, 33 and 40 represent dilution of lactate due to culture feeding (partial medium exchanges). Likewise, no difference was seen between test groups with respect to lactate production (FIG. 3).

Microscopic examination revealed that tumor cultures were successfully established in all six of the RealBio D4™ Culture Systems seeded with dissociated tumor tissue regardless of the variation of scaffold material or orientation. Observations on day 19 revealed an even distribution of cells across the top of scaffolds in Test Groups 1 and 2 with less than full scaffold coverage. The distribution of cells across the top of the scaffold from Test Group 3 was uneven, with some densely covered areas and some areas essentially devoid of cells. The underside of the scaffolds from all test groups was sparsely populated on Day 19. By Day 42, the top and bottom surfaces of scaffolds from all test groups were completely covered with cells. HEMA 3 staining revealed multiple cell types in all cultures.

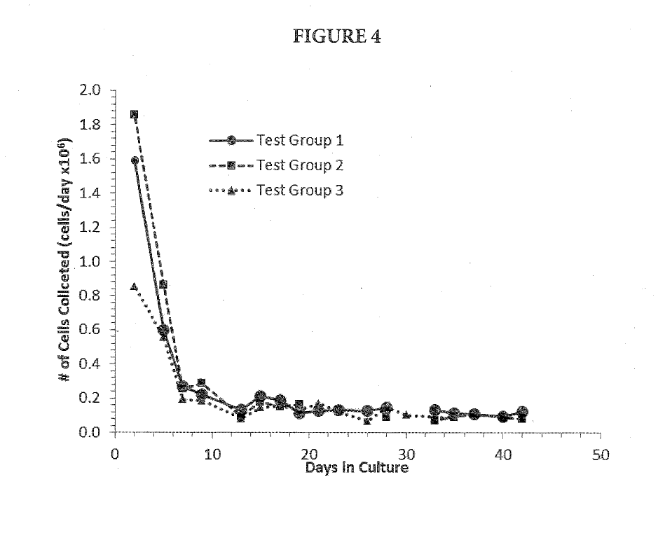

The total number of cells migrating from the cultures in each test group was evaluated 3× per week and normalized with respect to the number of days between sampling (FIG. 4). For FIG. 4, the number of cells harvested from the lower compartment of each bioreactor was normalized on a per day basis for each test group. Values for time points through Day 19 represent the average from duplicate cultures while values from time points beyond Day 19 are from a single culture only. Gaps in each profile represent cell numbers below the threshold the cell counting instrument. After the first week in culture, the migration rates stabilized at fewer than 2×10⁵ cells/day for each test group.

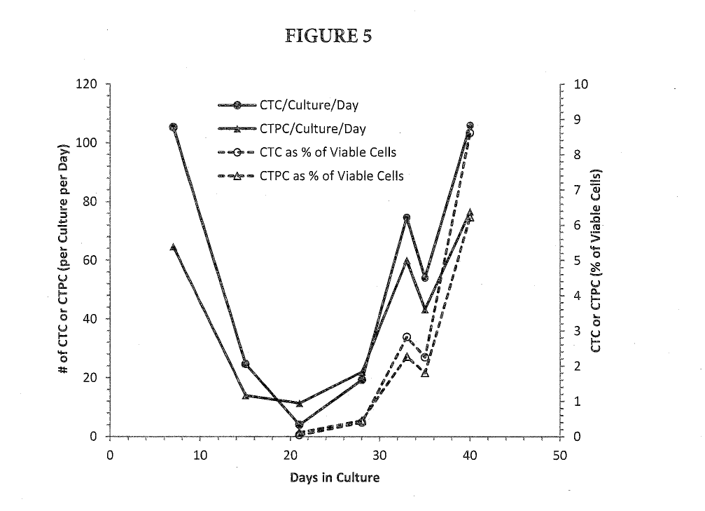

For comparison, the analysis of CTCs and CTPCs in mouse tissues (whole blood, 0.8 mL, and the dissociated tumor suspension used to seed the bioreactors, 0.5 mL) is shown in Table 3. The number of CTCs and CTPCs collected from the lower compartment of tumor cultures across the duration of the study is detailed in Table 4. The rate of CTC and CTPC production is shown in FIG. 5. On FIG. 5, the mean sum of CTC and CTPC cell counts (excluding clusters) across the three test groups is shown after being normalized to account for the number of cultures sampled at each time point and the number of days between sample collections, and as a percentage of the total viable cell count. On each plot, the first four data points represent samples that were frozen prior to analysis and exhibited poor viability, while the last three data points represent samples processed without freezing resulting in significantly higher viability.

TABLE 3
CTC [symbol] CTPC Analysis of Mouse Tissue Samples

| | | CTC | | CTPC |
|---|---|---|---|---|
| Sample | CTCs | Clusters | CTPC | Clusters |
| Blood (0.8 mL) | 30 | 0 | 16 | 2 |
| Dissociated | 20 | 0 | 16 | 0 |
| Tumor (0.5 mL) | | | | |

Fresh blood sample (not frozen) was analyzed ˜48 hours after collection. <1% of tumor cells were viable upon thawing prior to analysis.

TABLE 4
CTC [symbol] CTPC Analysis of Cells Migrating from Tumor Cultures

| | | Cell Count | | CTC | | CTPC |
|---|---|---|---|---|---|---|
| Test | | (Viable/ | | Clus- | | Clus- |
| Group | Sample | Total) | CTCs | ters | CTPCs | ters |
| 1 | Day 2, 5, 7 | <1%/nd | 70 | 4 | 44 | 0 |
| | Pool^{1,2} | | | | | |
| | Day 9, 13, | <1%/nd | 44 | 2 | 20 | 0 |
| | 15 Pool^{1,2} | | | | | |
| | Day 17, 19, | 1360/12,600 | 0 | 0 | 4 | 2 |
| | 21 Pool^{1,2} | | | | | |
| | Day 23, 26, | 1340/10,800 | 26 | 2 | 30 | 4 |
| | 28 Pool¹ | | | | | |
| | Day 33 | 2,230/2,800 | 32 | 0 | 40 | 0 |
| | Day 35 | 1,700/8,500 | 54 | 0 | 46 | 0 |
| | Day 40 | 190/1,000 | 74 | 0 | 44 | 0 |
| 2 | Day 2, 5, 7 | <1%/nd | 166 | 0 | 76 | 2 |
| | Pool^{1,2} | | | | | |
| | Day 9, 13, | <1%/nd | 18 | 0 | 16 | 0 |
| | 15 Pool^{1,2} | | | | | |
| | Day 17, 19, | 880/2,600 | 6 | 0 | 10 | 0 |
| | 21 Pool^{1,2} | | | | | |
| | Day 23, 26, | 740/3,000 | 0 | 0 | 28 | 4 |
| | 28 Pool¹ | | | | | |
| | Day 33 | 2,450/2,800 | 52 | 0 | 48 | 0 |
| | Day 35 | 1,420/3,500 | 62 | 0 | 50 | 0 |
| | Day 40 | 740/2,000 | 84 | 2 | 64 | 4 |
| 3 | Day 2, 5, 7 | <1%/nd | 80 | 0 | 74 | 0 |
| | Pool^{1,2} | | | | | |
| | Day 9, 13, | <1%/nd | 12 | 0 | 6 | 0 |
| | 15 Pool^{1,2} | | | | | |
| | Day 17, 19, | 340/15,600 | 6 | 2 | 20 | 2 |
| | 21 Pool^{1,2} | | | | | |
| | Day 23, 26, | 640/10,200 | 32 | 2 | 8 | 0 |
| | 28 Pool¹ | | | | | |
| | Day 33 | 3,350/3,500 | 140 | 0 | 92 | 0 |
| | Day 35 | 980/2,000 | 46 | 0 | 34 | 0 |
| | Day 40 | 280/1,500 | 160 | 0 | 122 | 0 |

¹Individual samples were frozen and thawed prior to pooling.
²Samples were collected from duplicate cultures through Day 19 but single cultures thereafter.

### Example 3

Effect of Hypoxia on In Vitro Production of Circulating Tumor Cells (CTCs) by Metastatic Pancreatic Tumor Tissue

Metastatic pancreatic tumor tissue was obtained as a fresh mouse xenograft tumor (P1) from a commercial source. The xenograft tumor was originally derived from a stage IV metastatic adenocarcinoma of the pancreas that had metastasized to the peritoneum of a 78 year old male patient. Upon receipt, the tumor was processed by mechanical and partial enzymatic dissociation and the RealBio D4™ Culture System bioreactors were seeded essentially as described in Example 2. Duplicate bioreactors were prepared for each of four test groups differing only with respect to the concentration and mode of oxygen delivery (Table 5).

TABLE 5
RealBio D4 ™ Culture System Bioreactor Configurations

| Test Group | Cell Type | Gas Supply |
|---|---|---|
| 1 | Primary | Passive, Normoxia |
| | Tumor | |
| 2 | Primary | Active, Normoxia |
| | Tumor | |
| 3 | Primary | Active, Moderate Hypoxia |
| | Tumor | |
| 4 | Primary | Active, Normoxia (first 20 Days) |
| | Tumor | Active, Moderate Hypoxia (last 31 |
| | | Days) |

Passive delivery of oxygen at the ambient concentration (˜21%) was accomplished by placing culture chambers in a standard, humidified CO₂ incubator at 37° C. with 5% CO₂. Active delivery of oxygen at either 2 or 20% was accomplished by perfusing humidified premixed gas (2% O₂/5% CO₂/93% N₂ or 20% O₂/5% CO₂/75% N₂). No difference was observed in the dissolved oxygen concentrations measured in culture systems configured with active gassing using 20% oxygen or passive ambient gassing (measured dissolved oxygen concentration≈21% in each case, i.e., normoxia). The concentration of dissolved oxygen was determined using an ISO₂ dissolved oxygen meter (World Precision Instruments, Inc., Sarasota, Fla.).

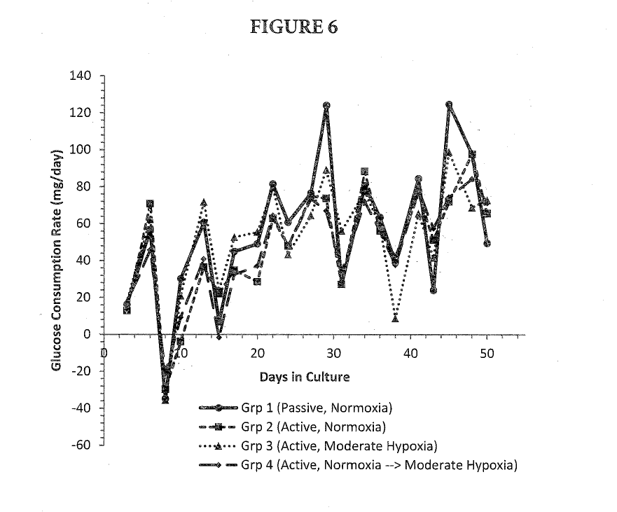

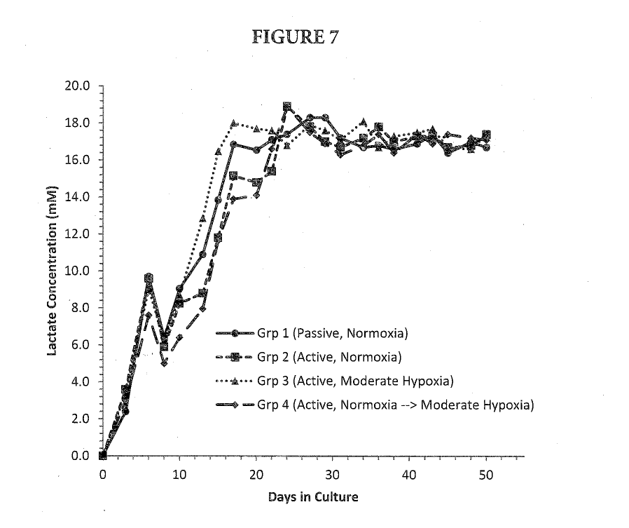

To assess the progress of the cultures, glucose and lactic acid concentrations were determined and the total cell density of samples collected from the bioreactor was estimated essentially as described in Example 2. Results are shown in FIGS. 6-7. Glucose consumption rates trended upwards for all cultures across the duration of the study, although significant fluctuations in glucose consumption rates that correlated with routine feeding intervals were present (FIG. 6). The steady state lactate concentration of 18 mM was observed for all tumor cultures (FIG. 7). Note the relatively smooth and consistent profiles observed for all test groups despite significant lactate dilution as a result of partial medium exchanges (feeding) on days 3, 10, 17, 24, 27, 31, 38 and 45. The cultures from Test Groups 1 and 3 (passive ambient oxygen delivery and moderately hypoxic conditions, respectively) demonstrated consistently higher glucose consumption rates and lactate concentration between days 10 and 20 compared to the other cultures suggesting that there was a genuine enhancement of metabolic rates during this portion of the study. These differences disappeared, however, during the latter half of the study as the glucose consumption and lactate production rates for Test Groups 2 and 4 (active oxygen delivery and active oxygen delivery followed by hypoxia, respectively) rose to meet those of Groups 1 and 3 in the final 30 days of the study.

One culture from each test group was terminated after 20 days and the remaining culture after 51 days. Upon termination, the fabric scaffold was excised from each bioreactor and divided into sections for examination of tissue development by direct staining and scanning electron microscopy essentially as described in Example 2.

Microscopic examination revealed that the concentration of oxygen during the first 19 days of the study affected the distribution of cells across the culture scaffold. Large, dense “colonies” of cells with sparse cell populations in between were observed across the upper surface of the scaffolds from all of the cultures exposed to normoxia (passive or active delivery) while a more uniform cell distribution was observed across the scaffold from the culture that was maintained under moderately hypoxic conditions. The underside of the scaffolds from all test groups was sparsely populated. These distribution patterns persisted through the end of the study (51 days).

Identification and enumeration of CTCs and CTPCs was performed essentially as described in Example 2. Results are shown in Table 6.

TABLE 6
CTC [symbol] CTPC Analysis of Cells Migrating from Tumor Cultures

| | | Cell Count | | | | |
|---|---|---|---|---|---|---|
| Test | | (Viable/ | | CTC | | CTPC |
| Group | Sample | Total) | CTCs | Clusters | CTPC | Clusters |
| 1 | Day 20 | 2700/7560 | 92 | 0 | 96 | 0 |
| Passive, | Day 20 | 2100/5400 | 50 | 0 | 34 | 0 |
| Normoxia | Day 27 | 230/840 | 6 | 0 | 2 | 0 |
| | Day 34 | 180/1400 | 0 | 0 | 4 | 2 |
| | Day 41 | 260/1960 | 8 | 0 | 12 | 0 |
| 2 | Day 20 | 610/1400 | 2 | 0 | 2 | 0 |
| Active, | Day 20 | 770/1680 | 4 | 0 | 0 | 0 |
| Normoxia | Day 27 | 140/360 | 4 | 0 | 2 | 0 |
| | Day 34 | 250/1960 | 22 | 0 | 30 | 0 |
| | Day 41 | 980/2800 | 24 | 0 | 68 | 0 |
| 3 | Day 20 | 1700/3640 | 2 | 0 | 6 | 0 |
| Active, | Day 20 | 510/2800 | 4 | 0 | 0 | 0 |
| Moderate | Day 27 | 150/280 | 0 | 0 | 0 | 2 |
| Hypoxia | Day 34 | 170/1120 | 20 | 0 | 8 | 0. |
| | Day 41 | 440/2520 | 6 | 4 | 6 | 0 |
| 4 | Day 20 | 600/3360 | 2 | 0 | 4 | 0 |
| Active, | Day 20 | 550/2800 | 0 | 0 | 0 | 0 |
| Normoxia → | Day 27 | 80/280 | 8 | 0 | 2 | 2 |
| Moderate | Day 34 | 120/1400 | 12 | 0 | 4 | 0 |
| Hypoxia | Day 41 | 290/1960 | 14 | 0 | 6 | 4 |

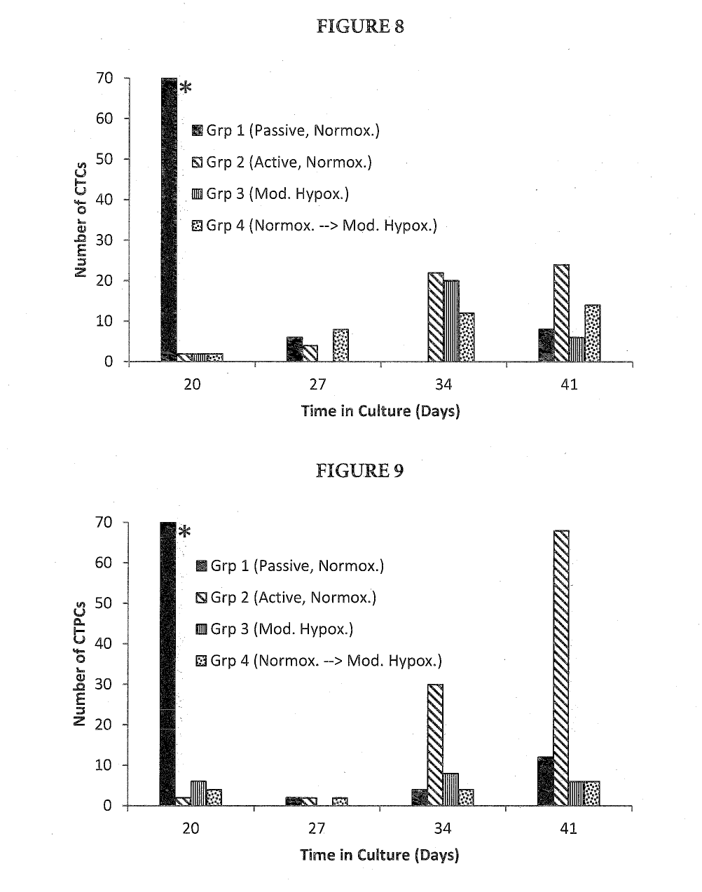

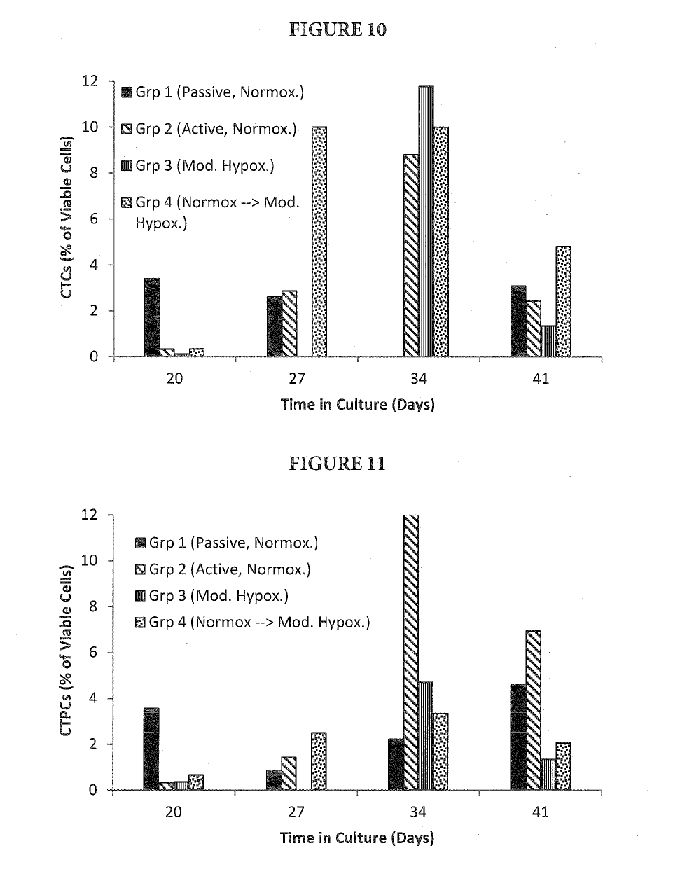

The number of cells migrating from the tumor cultures in each test group was evaluated 3× per week and normalized with respect to the number of days between sampling. The migrating cell numbers fluctuated around 4−5×10⁴ cells per day for each of the test groups with no obvious correlation with oxygen levels. The numbers of CTCs and CTPCs was also evaluated (FIGS. 8-9). The asterisk for Group 1, Day 20 signifies a more aggressive sampling technique used for that single sample (value off scale). Oxygen had no clear effects on the number of migrating cells expressing the CTC and CTPC phenotype, though the samples from Test Group 2 (active delivery, normoxia) demonstrated the highest number of CTCs and CTPCs during the last two sampling intervals tested. There was no difference in the fraction of CTCs, however, the fraction of CTPCs was elevated in Test Group 2 (FIGS. 10-11).

### Example 4

### Effect of Hypoxia on In Vitro Production of Circulating Tumor Cells (CTCs) by Pancreatic Cell Lines

Two human pancreatic cancer cell lines reported to be highly metastatic in mouse xenograft models (MIA PaCa-2 and AsPC-1) and two human pancreatic cancer cell lines that rarely metastasize in mouse xenograft models (PL45 and Capan-2) were obtained from the American Type Culture Collection (ATCC). Each cell line was maintained in T-75 flasks at 37° C., 5% CO₂ using the growth medium recommended by the ATCC prior to culturing in RealBio D4™ Culture Systems. The RealBio D4™ Culture System bioreactors were prepared and seeded essentially as described in Example 2. The bioreactors were configured as described in Table 7. Two bioreactors were prepared for culturing each of the four cell lines (8 systems total).

TABLE 7
RealBio D4 ™ Culture System Bioreactor Configurations-Cell Line Cultures

| Cell Type | Metastatic Potential | Growth Medium |
|---|---|---|
| AsPC-1 | High | RPMI 1640, 10% FBS |
| Capan-2 | Low | McCoy's 5a Medium, 10% FBS |
| PL45 | Low | DMEM, 10% FBS |
| MIA PaCa-2 | High | DMEM, 10% FBS, 2.5% Horse Serum |

One culture from each test group was terminated after 13 days, while the remaining culture from each test group was terminated after 39 days. Upon termination, the fabric scaffold was excised from each culture chamber and divided into sections for examination of tissue development by direct staining essentially as described in Example 2.

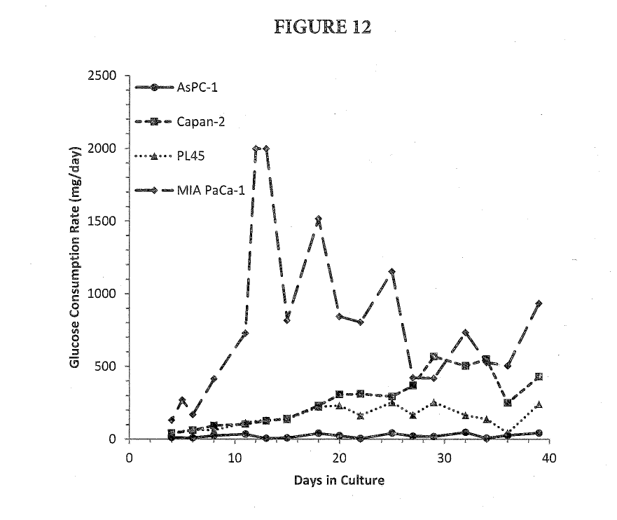

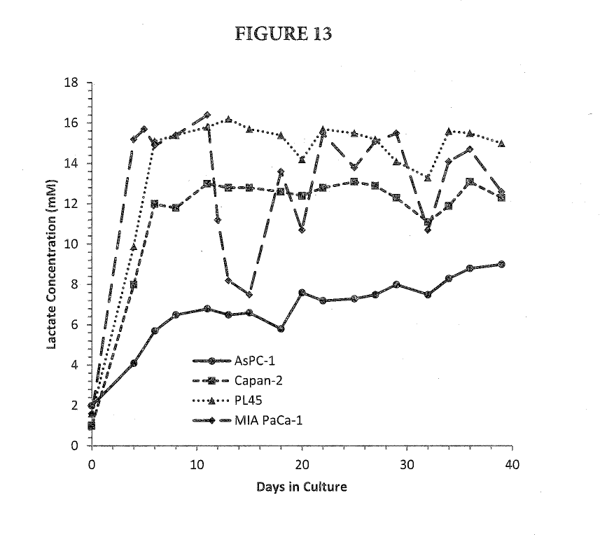

To assess the progress of the cultures, glucose and lactic acid concentration was determined and the total cell density of cell cultures periodically collected from the bioreactors was estimated essentially as described in Example 2. A peak glucose consumption rate of approximately 2000 mg/day was observed for the MIA PaCa-2 culture after only 2 weeks of incubation. This rate of glucose utilization was nearly four times that of the next most active culture (Capan-2) and approximately fifty times higher than that observed for the AsPC-1 culture (FIG. 12). Despite the wide disparity in glucose consumption rates for the four cell lines, the steady state lactate levels varied by less than a factor or two (FIG. 13). The irregularity of the curves for the MIA PaCa-2 cell line in FIGS. 12 and 13 may relate in part to differences in the glucose concentration of the medium supplied to the culture at different times throughout the study.

Microscopic examination of culture scaffold sections removed from the culture chambers was performed after 13 and 39 days. MIA PaCa-2 cells expanded rapidly in the culture chambers, covering the upper surface of the scaffold fabric with multiple cell layers and occluding most of the large voids between scaffold fiber bundles by Day 13, though the underside of the scaffold remained largely unpopulated. By Day 39, very heavy accumulations of the cells were found on top of the scaffold material along with moderate to heavy cell densities on the underside. Essentially all of the cells exhibited a rounded morphology whether they are found attached directly to scaffold fibers or associated with other cells in dense, tissue-like clusters, and although most of the cells were adherent, a large number of cells could be seen sloughing off the culture scaffold when the medium was drained from the culture chamber for histological examination on Day 39.

AsPC-1 cultures exhibited moderate cell densities and significant amounts of natural extracellular matrix material across the top of the scaffold fabric after 13 days in culture, and though cell densities increased only modestly after Day 13, many of the larger voids between the fiber bundles of the scaffold fabric were filled with cells by the time that cultures were terminated on Day 39. The underside of the culture scaffold, however, remained essentially devoid of cells for the duration of the study. The size and shape of AsPC-1 cells appeared more heterogeneous compared to the relatively uniform morphology of MIA PaCa-2 cells.

Capan-2 and PL45 cells arranged themselves in very similar fashion on the scaffold material with all cells found very closely associated with scaffold fibers and very few cells spanning open areas between fibers. After 13 days in culture, both of these cell types covered a majority of the scaffold fiber bundles on the upper side of the scaffold but the large “pores” between fiber bundles remained open. The density of cells on the fiber bundles was higher after 39 days but the vast majority of scaffold “pores” still remained open and only spotty “ribbons” of cells closely associated with scaffold fibers were observed on the underside of the scaffold. It appeared that the Capan-2 culture exhibited slightly higher cell densities overall when compared to the PL45 cell line. Interestingly, no isolated single cells could be found in the Capan-2 culture (only very few were observed in the PL45 cultures) and neither the Capan-2 nor the PL45 cells produced visible amounts of natural extracellular matrix material.

Identification and enumeration of CTCs and CTPCs was performed essentially as described in Example 2. Results are shown in Table 8.

TABLE 8
CTC [symbol] CTPC Analysis of Cells Migrating from Pancreatic Cell Lines
Maintained in the RealBio D4 ^{™} Culture System Bioreactors

| | | Cell Count | | | | |
|---|---|---|---|---|---|---|
| Cell | | (Viable/ | | CTC | | CTPC |
| Line | Sample | Total) | CTCs | Clusters | CTPC | Clusters |
| AsPC-1 | Day 25 | 7200/25000 | 1010 | 0 | 1080 | 4 |
| | Day 32 | 5000/8400 | 306 | 0 | 404 | 0 |
| Capan-2 | Day 25 | 8600/56000 | 16 | 0 | 22 | 0 |
| | Day 32 | 13600/28000 | 24 | 0 | 22 | 0 |
| PL45 | Day 25 | 1640/45000 | 50 | 0 | 16 | 0 |
| | Day 32 | 880/4200 | 16 | 0 | 10 | 0 |
| MIA | Day 25 | 80000/365000 | 18196 | 0 | 16000 | 0 |
| PaCa-2 | Day 32 | 6000/30000 | 1040 | 0 | 1260 | 0 |

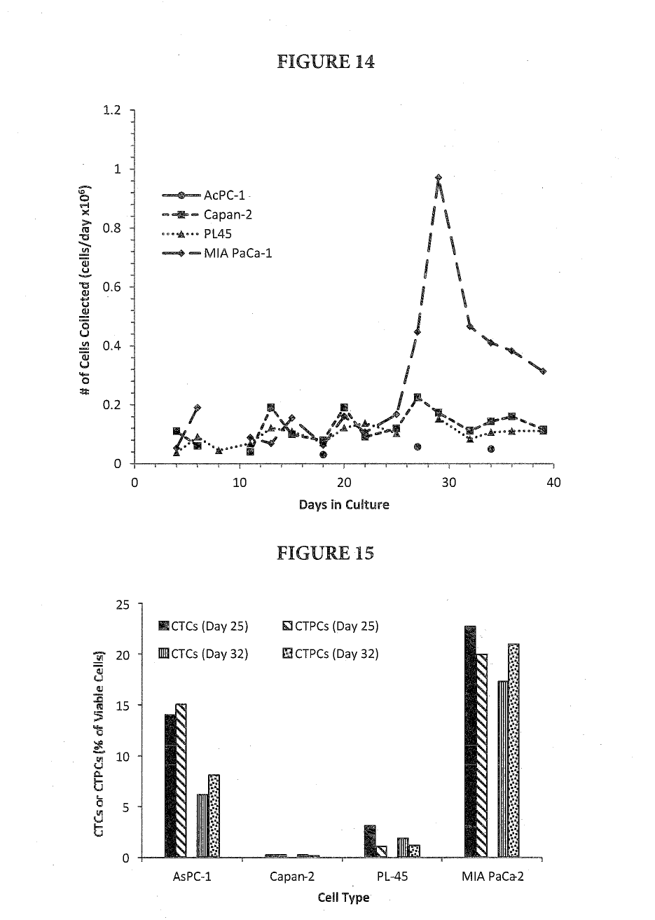

FIG. 14 illustrates the number of cells harvested from the lower compartment of each bioreactor and normalized on a per day basis for each pancreatic cancer cell line. Gaps in each profile represent cell numbers below the threshold of the cell counting instrument. The number of cells migrating out of the cell line cultures into the bottom compartment of the bioreactor chambers did not appear significantly different for any of the cell lines until a burst of cells was produced by the MIA PaCa-2 cell line beginning after 25 days in culture. The relative rate of CTC and CTPC production is shown on FIG. 15.

### Example 5

Comparison of in vitro CTC Production by Metastatic and Non-metastatic Cell Lines Grown in the RealBio D4™ Culture System Bioreactor under Normoxia and Hypoxia

Two human pancreatic cancer cell lines described in Example 4: highly metastatic MIA PaCa-2 and rarely metastatic Capan-2 were used. Four bioreactors were prepared for culturing each cell line (8 bioreactors total). The bioreactors were prepared and seeded essentially as described in Example 2. The configuration of bioreactors is shown in Table 9.

TABLE 9
RealBio D4 ™ Culture System Bioreactor Configurations

| Test Group | Cell Type | Metastatic Potential | Gassing Condition |
|---|---|---|---|
| 1 | Capan-2 | Low | Normoxia |
| 2 | Capan-2 | Low | Hypoxia |
| 3 | MIA PaCa-2 | High | Normoxia |
| 4 | MIA PaCa-2 | High | Hypoxia |

Passive delivery of oxygen at the ambient concentration (˜21%) was maintained for the first 10 days of the study by keeping culture chambers in a standard, humidified CO₂ incubator at 37° C. with 5% CO₂. Beginning on Day 10, delivery of oxygen to the cultures was initiated and performed essentially as described in Example 3. On Day 13, the flow of medium was changed for all cultures from pulsed flow to constant flow at a slower rate (maintaining the overall medium exchange rate within the culture chambers).

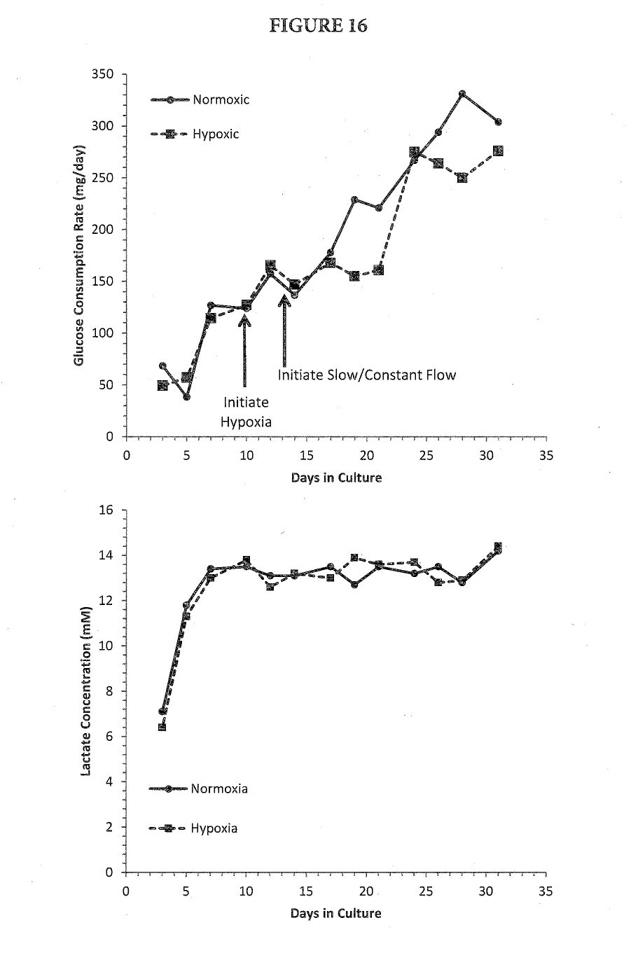

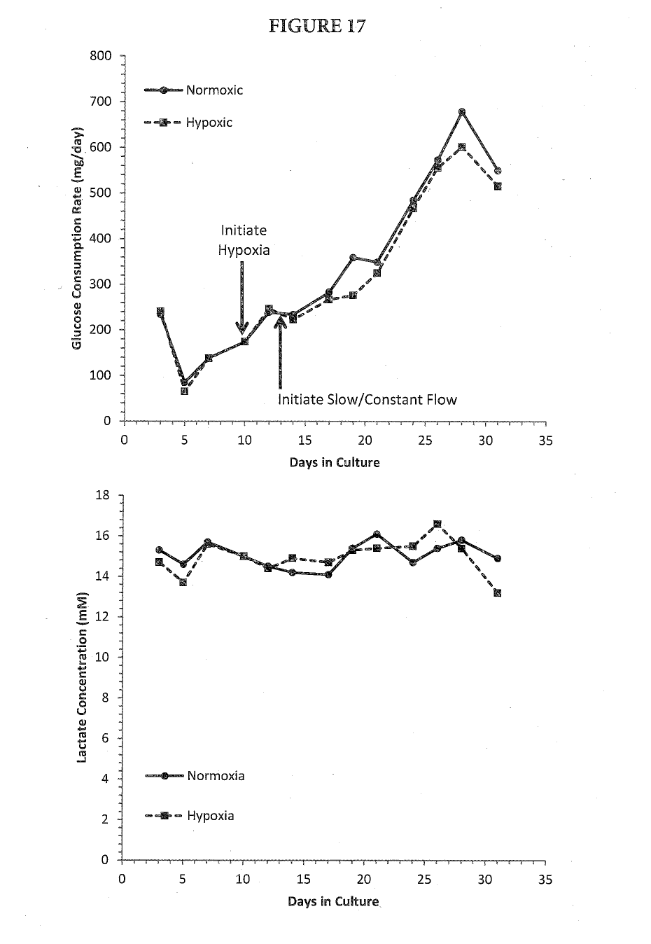

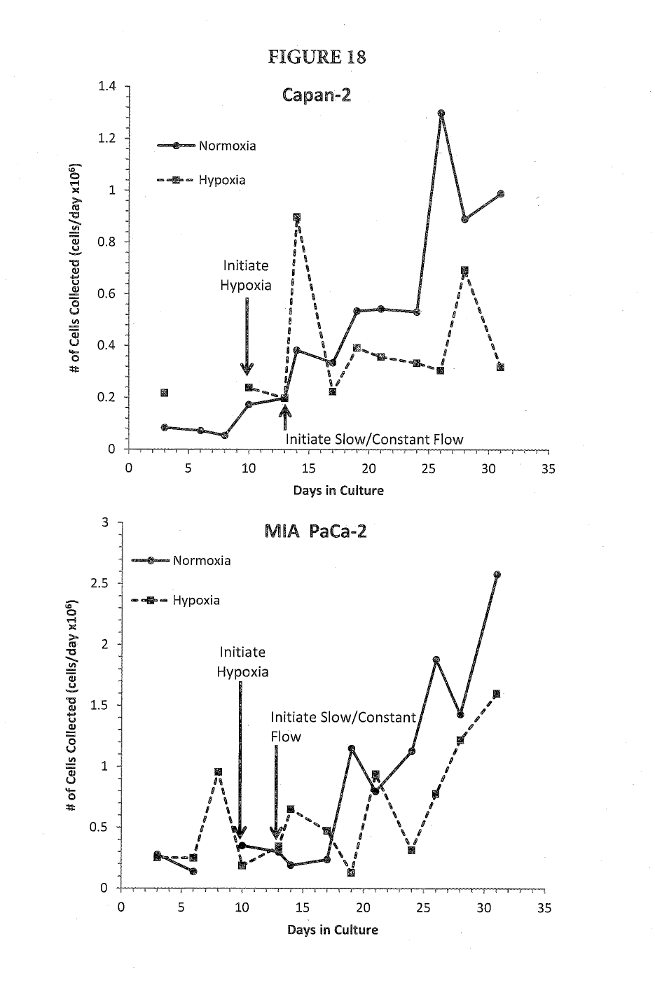

To assess the progress of the cultures, glucose and lactic acid concentration, and oxygen concentration in the medium were determined and the total cell density was estimated essentially as described in Example 2. Glucose consumption rates trended upwards throughout the course of the study for both cell lines (FIGS. 16-17). Neither cell line exhibited obvious changes in lactate production when switched from normoxic to hypoxic conditions. Note the relatively smooth and consistent lactate profiles observed despite significant lactate dilution as a result of partial medium exchanges (feeding) performed at each sampling interval beginning on Day 5. FIG. 18 illustrates the number of cells harvested from the lower compartment of each bioreactor and normalized on a per day basis for each cell line. Gaps in each profile represent cell numbers below the threshold of the cell counting instrument. The switch to hypoxia caused an apparent decrease in the total number of cells migrating out of the cultured cell masses for both cell lines.

Samples were collected from the bottom compartment of each bioreactor and partial medium exchanges were performed 3 times per week. One culture from each test group was terminated after 18 days, and the remaining culture after 31 days. Upon termination, the fabric scaffold was excised from each culture chamber and examined for tissue development by direct staining essentially as described in Example 2.

Identification and enumeration of CTCs and CTPCs was performed essentially as described in Example 2. Results are shown in Table 10.

TABLE 10
CTC [symbol] CTPC Analysis of Cells Migrating from 31-Day Cultures

| | Gassing | CTCs | CTPCs |
|---|---|---|---|
| Cell Type | Condition | (% of viable cells) | (% of viable cells) |
| Capan-2 | Normoxia | 16 (0.1%) | 12 (0.1%) |
| | Hypoxia | 8 (0.1%) | 59 (0.9%) |
| MIA PaCa-2 | Normoxia | 764 (4.2%) | 1520 (8.3%) |
| | Hypoxia | 712 (4.0%) | 620 (3.5%) |

Microscopic examination revealed that under normoxia (20% oxygen), the density and arrangement of Capan-2 cells was uniform across the entire culture scaffold. Under hypoxic conditions, the arrangement of cells varied across the length of the culture scaffold. The varied growth pattern gradient observed across the scaffold suggests the presence of an oxygen gradient across the scaffold and therefore a mixture of cell cells growing under varying levels of hypoxia. For MIA PaCa-2 cells no obvious difference was observed in cell morphology or cell arrangements. For both cell lines, the density was notably lower under hypoxic conditions.

While the invention has been described in detail with reference to specific examples, it will be apparent to one skilled in the art that various modifications can be made within the scope of this invention. Thus the scope of the invention should not be limited by the examples described herein, but by the claims presented below.

We claim:

1. A combination of a bioreactor and one or more tumor cells in which the system parameters are such that the tumor cells maintain their normal metastatic potential.

2. The combination of claim 1, wherein the bioreactor comprises a cell-supporting but cell-permeable matrix separating at least two fluid chambers with fluid flowing therethrough and at least one gas chamber connected to each of the fluid chambers.

3. The combination of claim 1, wherein said one or more tumor cells have been introduced into one of the at least two fluid chambers containing suitable nutrient medium and gas sufficient to sustain tumor growth.

4. The combination of claim 1, wherein the normal metastatic potential comprises the ability to produce metastatic cells.

5. The combination of claim 4, wherein the metastatic cells include cells having characteristics of circulating tumor cells and circulating tumor progenitor cells.

6. The combination of claim 5, wherein the characteristics of circulating tumor cells and circulating tumor progenitor cells include the presence of one or more of the following biomarkers: EpCAM, CK5, CK7, CK18, CK19, Cd44v6, EphB4, FAP (seprase), IGF-1R, BCL2, HER2, CA19-9, CEA, CD133, MUC1, N-cadherin, Survivin and PTEN.

7. A method of generating metastatic tumor cells in vitro comprising:
(a) introducing one or more tumor cells into a bioreactor;
(b) providing to the bioreactor fluid culture media and gas composition supportive of growth of the tumor cells;
(c) incubating the bioreactor under conditions and for a time sufficient for the tumor cells to produce metastatic cells;
(d) collecting metastatic tumor cells.

8. The method of claim 7, wherein the bioreactor comprises a cell-supporting but cell-permeable matrix separating at least two fluid chambers with fluid flowing therethrough and at least one gas chamber connected to each of the fluid chambers.

9. The method of claim 7, wherein the tumor cells are introduced in the first fluid chamber and fluid culture media and gas composition supportive of tumor growth are supplied to said at least two fluid chambers.

10. The method of claim 7, wherein conditions including one or more of glucose concentration, lactic acid concentration and pH are monitored in the bioreactor during step (c).

11. The method of claim 7, wherein metastatic tumor cells are collected from among cells that have migrated into said second chamber.

12. The method of claim 7, further comprising confirming the nature of metastatic tumor cells by detecting the presence of one or more of the following biomarkers: EpCAM, CK5, CK7, CK18, CK19, Cd44v6, EphB4, FAP (seprase), IGF-1R, BCL2, HER2, CA19-9, CEA, CD133, MUC1, N-cadherin, Survivin and PTEN

13. A method of manipulating a culture of tumor cells in a bioreactor to reveal or alter metastatic potential of the tumor cells.

14. The method of claim 13, wherein the bioreactor comprises a cell-supporting but cell-permeable matrix separating at least two fluid chambers with fluid flowing therethrough and at least one gas chamber connected to each of the fluid chambers.

15. The method of claim 13, wherein said tumor cells have been introduced into one of the at least two fluid chambers containing suitable nutrient medium and gas sufficient to sustain tumor growth.

16. The method of claim 13, wherein manipulating the culture comprises altering of one or more of suitable nutrient concentration, oxygen concentration and acidity.

17. The method of claim 13, wherein manipulating the culture comprises administering one or more of test compounds, antibodies or biologics.

18. The method of claim 13, wherein the metastatic potential is measured by the numbers of cells produced in the bioreactor that possess one or more of the following biomarkers: EpCAM, CK5, CK7, CK18, CK19, Cd44v6, EphB4, FAP (seprase), IGF-1R, BCL2, HER2, CA19-9, CEA, CD133, MUC1, N-cadherin, Survivin and PTEN.

19. A method of identifying an agent capable of inhibiting or stimulating tumor metastasis comprising:
(a) preparing a suspension of cells derived from a tumor;
(b) introducing a sample of the suspension into a first fluid chamber of a bioreactor, the bioreactor, comprising a cell-supporting but cell-permeable matrix separating at least two fluid chambers with fluid flowing therethrough and at least one gas chamber connected to each of the fluid chambers;
(c) supplying to said at least two fluid chambers fluid culture media and gas composition suitable to support tumor growth;
(d) incubating the bioreactor under conditions and for a time sufficient for cell proliferation and production of metastatic cells;
(e) introducing a candidate agent into the first or the second chamber;
(f) collecting cells that have migrated into the second chamber;
(g) identifying and monitoring the fraction of metastatic tumor cells among the cells collected in step (f).

20. The method of claim 19, wherein the agent is selected from a small-molecule compound, an antibody or a biologic.

21. The method of claim 19, wherein conditions including one or more of glucose concentration, lactic acid concentration and pH are monitored in the bioreactor during steps (d)-(e).

22. The method of claim 19 wherein the metastatic cells are identified by the presence of one or more of the following biomarkers: EpCAM, CK5, CK7, CK18, CK19, Cd44v6, EphB4, FAP (seprase), IGF-1R, BCL2, HER2, CA19-9, CEA, CD133, MUC1, N-cadherin, Survivin and PTEN.

23. A method of assessing metastatic potential of a tumor comprising:
(a) preparing a suspension of cells derived from the tumor;
(b) introducing a sample of the suspension into a first fluid chamber of a bioreactor, the bioreactor, comprising a cell-supporting but cell-permeable matrix separating at least two fluid chambers with fluid flowing therethrough and at least one gas chamber connected to each of the fluid chambers;
(c) supplying fluid culture media and gas composition supportive of tumor growth to said at least two fluid chambers;
(d) incubating the bioreactor under conditions and for a time sufficient for cell proliferation and production of metastatic cells;
(e) collecting cells that have migrated into the second chamber;
(f) identifying metastatic tumor cells among the cells collected in step (e).

24. The method of claim 23, further comprising assessing the fraction of metastatic tumor cells among the cells collected in step (e).

25. The method of claim 23, wherein the metastatic cells in step (f) are identified by the presence of one or more of the following biomarkers: EpCAM, CK5, CK7, CK18, CK19, Cd44v6, EphB4, FAP (seprase), IGF-1R, BCL2, HER2, CA19-9, CEA, CD133, MUC1, N-cadherin, Survivin and PTEN.

26. The method of claim 23, wherein conditions including one or more of glucose concentration, lactic acid concentration and pH are monitored in the bioreactor during steps (d).
